(19) United States
(12) Patent Application Publication
(10) Pub. No.: US 2021/0244497 A1
(43) Pub. Date: Aug. 12, 2021

(54) Stoma Cleaning Apparatus and Method

(71) Applicant: Noor G Taweh, Farmington, CT (US)

(72) Inventor: Noor G Taweh, Farmington, CT (US)

(21) Appl. No.: 16/783,222

(22) Filed: Feb. 6, 2020

(51) Int. Cl. A61B 90/80; A61F 5/445

(52) U.S. Cl. CPC A61B 90/80; A61F 5/445

(57) ABSTRACT

An apparatus for restraining and/or containing a stoma during cleaning and disinfection of an area, and in particular the skin area, around a stoma in preparation for attaching an ostomy bag or appliance.

### TECHNICAL FIELD

The present disclosure generally relates to an apparatus to aid in preparing a user's body for attaching an ostomy bag or stoma pouch, and particularly relates to an apparatus for restraining and/or containing a stoma during cleaning and disinfection of areas, such as the skin area, around the stoma.

### BACKGROUND

The number of people worldwide with a stoma is nearly 2 million. In the United States of America, according to the United Ostomy Association of America, there are over 750,000 Americans with at least one kind of stoma. Likewise, there are over 100,000 new ostomy surgeries per annum. During such surgeries a stoma is created, such as a stoma on the neck for breathing. In most cases, a stoma is an opening on the abdomen created by the ostomy surgery, i.e., when a portion of the colon or the rectum is removed and the remaining colon is brought to the abdominal wall. There are several types of stoma depending on medical procedure used to create them—e.g., colostomy, urostomy, and/or ileostomy. Surgical procedures that involve the creation of an artificial stoma have names that typically end with the suffix “-ostomy”, and the same names are also often used to refer to the stoma thus created. For example, the word “colostomy” often refers either to an artificial anus or the procedure that creates one. Accordingly, it is not unusual for a stoma to be called an ostomy (plural ostomies). For most ostomies, a pouch is worn over the stoma to collect stool or urine. A small pouch, or ostomy/colostomy bag, is then placed over the stoma to collect waste products that would normally pass through a person's rectum and anus.

Typical ostomy appliances include a pouch (e.g., ostomy or colon ostomy bag) that your stoma drains into. Ostomy appliances may be of various constructions including one-piece bags with an integral adhering wafer (such as the Hollister 8553 Premier One-Piece Pouch) and two-piece appliances (such as the Coloplast Sensura Mio Flex Two-Piece Pouch). Depending on the type of pouch and user habits, a user will need to change it periodically (e.g., every three to seven days). When a user changes/removes the pouch, the skin around the stoma is generally cleaned with warm water and/or other solutions to remove adhesives and soil to create a dry and disinfected surface for application of an ostomy device. During the process of cleaning, the user must assure that the stoma and skin area surrounding the stoma are clean and disinfected, and that the cleaned skin area provides an adherent surface condition for attaching an ostomy appliance.

In particular, having a stoma means that the user has no control over defecation. Unforeseen defecation may cause re-soiling and infection of an area (particularly the skin area) around the stoma and require the user to reclean the area. Continual cleaning/re-cleaning of the skin area around the stoma creates numerous discomforts for the user, for example skin irritation. In addition, exposure of the stoma to a/the non-sterile environment provides a high probability of infection agents reaching the stoma itself. Despite discomforts, the skin area around the stoma must be cleaned and kept clean to provide the ideal surface condition of the skin to assure the stoma appliance (e.g., stoma bag) may be fitted and adhered properly, particularly to avoid leakage.

In addition, for a period of time after ostomy surgery, patients experience soreness of the abdominal muscles and skin surrounding their stoma. In such cases, patients may be advised to lie on their backs with the torso and head propped up with pillows to alleviate any discomfort; however, for immobile patients and those lying on their backs, it is more difficult to control leaking stool.

The prior art largely ignores apparatuses for restraining and/or containing a stoma during cleaning and disinfection of the skin area around the stoma in preparation for attaching an ostomy bag or stoma pouch. Emphasis of prior inventions have been placed on different approaches—e.g., materials and structures, related to attaching ostomy devices to the body, such as the use of skin friendly adhesives, and/or, in the case of two-piece ostomy appliances, attaching bag holders to their respective bag collectors using various coupling devices and mechanisms.

Prior art references such as U.S. Pat. No. 6,409,710 to Holtermann and U.S. Pat. No. 8,142,406 to Blum, respectively, discuss the complexities, including physical and mental demands, experienced by users trying to attach ostomy appliances to their persons. As an example, they cite the need for ease of use, ease of handling, security of the bag affixed to the person, and leaktightness of appliances among other things. Holtermann discloses that the skin area surrounding the stoma is sensitive and sometimes painful, making application of ostomy appliances discomforting for users. Blum discusses methods for allowing a user to wear an ostomy appliance for longer periods of time. However, neither Holtermann or Blum, as a representative class of inventors, addresses issues of cleanliness, infection control, and appliance adherence to the skin particularly for when the ostomy appliance is removed from the body and skin area and must be replaced.

Other prior art references, for example, U.S. Pat. No. 9,707,120 to Nguyen-DeMary and Pat. Publs. US 2018/0000628 and WO 2019/198012 to Larsen and Santos, respectively, provide means for keeping a stoma and its surrounding skin area free of contamination utilizing controlled evacuation appliances to retain body waste inside the stoma tract, rings that orient or otherwise support the stoma within the attached ostomy bag, and/or fixtures that surround and contact the stoma face to control release of waste, respectively. Yet again, such means don't afford capture of waste from an active stoma when the ostomy device is being replaced.

In view of one or more problems stated above, it is desirable to have a hygienic apparatus available for use when changing/replacing an ostomy appliance that provides a user control over stoma evacuation while also allowing a user to clean and disinfect the skin area surrounding the stoma.

### SUMMARY OF THE DISCLOSURE

The present disclosure relates to an apparatus for restraining and/or containing a stoma during cleaning and disinfection of the skin area (or in the case of a two-piece ostomy appliance, where the wafer is not removed from the user's body, the wafer area) around the stoma in preparation for attaching an ostomy bag or stoma pouch (i.e., stoma-clean apparatus). In one embodiment the stoma-clean apparatus includes an absorbent, semi-transparent, flexible, lightweight waste collection tube (i.e., silo) with an open end and a closed end. The open end of the silo is elasticized in order to provide a means for holding the apparatus onto a stoma in a hands-free manner. In an aspect, the open end of the stoma-clean apparatus may include an elastic annular seal ring disposed on, or integrated with, the open end of the silo, configured to effectively cover, encase, and hold onto a stoma. In an aspect, the annular seal ring may be configured as an adjustable diameter clasp or other mechanism allowing adjustability of the inside diameter of the annular ring to allow the ring to cover, encase, and hold onto the stoma.

In a further embodiment, the stoma-clean apparatus includes an absorbent, semi-transparent, rigid/semi-rigid silo with a closed end and an open end, a rigid body with a thru-hole configured to enclose a stoma (i.e., cap), and a coupling ring for attaching the silo to the cap. In an aspect, the cap may be configured in a cylindrical shape with a flexible flanged end for contacting a portion of the skin area around a stoma.

In yet another embodiment, the stoma-clean apparatus includes an absorbent, semi-transparent, rigid/semi-rigid silo with a closed end and an open end, a cap, and a coupling ring for attaching the silo to the cap. In an aspect, the cap may be configured with a rigid/semi-rigid conical or spherical (hereinafter “conical/spherical”) end for contacting a portion of the skin area around a stoma. In another aspect, the cap may include a lip configured to allow a user to seat the cap while also utilizing the cap to apply pressure to a portion of the skin area around a stoma.

In still yet another embodiment, the stoma-clean apparatus may be configured as a one-piece unit including a silo, cap, and optionally a lip. A one-piece stoma-clean apparatus may be constructed from moldable, absorbent, and transparent medical grade materials such as those engineered by 3M, Adhesives Research, and Parafix Engineered Adhesives Solutions.

In accordance with the present disclosure, a stoma-clean apparatus allows a user of an ostomy/colostomy bag to replace an in-place bag by properly preparing the stoma and the skin area surrounding the stoma for application of another (“new”) bag. In various embodiments, the stoma-clean apparatus is configured for hand-held use, however, the apparatus may be incorporated with another device which is not hand-held, such as a waistband or belt.

In an embodiment, the stoma-clean apparatus includes an absorbent, semi-transparent, flexible, lightweight waste collection tube (i.e., silo) attached to an annular seal ring. The silo includes a closed end and an open end, wherein the open end is attached to the seal ring. The silo and seal ring assembly is operably placed over a stoma so as to contain any waste which may unexpectantly exit the stoma. Transparency of the silo allows a user to discern whether/if waste is exiting/exited from a stoma. The annular seal ring is of sufficient diameter to encase the outer periphery of the stoma at the surface of the user's body (i.e., skin area surrounding the stoma). The annular seal ring may be constructed of an elastic material which allows the ring to be elastically secured to the stoma. Such a construction allows for hands-free location and attachment of the ring to the stoma while providing sufficient, but minimal coverage of the skin area surrounding the stoma. With the stoma-clean apparatus seated on a minimal skin area surrounding the stoma, a user is able to clean and/or disinfect the skin area around the stoma.

In an aspect, the annular seal ring may be configured as an adjustable diameter clasp whereby the inside diameter of the seal ring may be adjusted for effectively securing the stoma-clean apparatus to the outside diameter of the stoma. In another aspect, the stoma-clean apparatus may include a coupling situated intermediate between the silo and the seal ring operatively configured to cooperate with the seal ring to vary its diameter.

In a further embodiment, the stoma-clean apparatus includes an absorbent, semi-transparent, rigid/semi-rigid silo with a closed end and an open end, a rigid body with a thru-hole configured to enclose a stoma (i.e., cap), and a coupling ring for attaching the silo to the cap. In an aspect of the present disclosure, the silo may be directly attachable to the cap. The silo and cap assembly is operably placed over a stoma so as to contain any waste which may unexpectantly exit the stoma. Transparency of the silo allows a user to discern whether/if waste is exiting/exited from a stoma. The cap is of sufficient inside diameter to encase the outer periphery of the stoma at the surface of the user's body (i.e., skin area surrounding the stoma). The cap is seated onto the skin area surrounding a stoma so as to provide sufficient, but minimal coverage of the skin area surrounding the stoma. With the stoma-clean apparatus seated on a minimal skin area surrounding the stoma, a user is able to clean and/or disinfect the skin area around the stoma.

The cap may be constructed of any suitable material and size which allows a user to effectively grasp the stoma-clean apparatus for locating it around a stoma and seating the cap on the skin area surrounding the stoma. In an aspect, the cap may be made of metals, alloys, or polymers, for example, which may be cleaned and/or disinfected so as to allow for reuse. In another aspect, the cap may be configured, at its distal end (i.e., end contacting the skin area), as a cylindrical, spherical, ellipsoidal, or other suitable shapes such that when the distal end of the cap contacts the skin area around the stoma it forms a seal between the cap and skin area whereby the exposed skin area around the stoma is of minimal size (e.g., diameter).

In yet another embodiment, the stoma-clean apparatus includes an absorbent, semi-transparent, rigid/semi-rigid silo with a closed end and an open end, a rigid body with a thru-hole configured to enclose a stoma (i.e., cap), and a coupling ring for attaching the silo to the cap. In an aspect of the present disclosure, the silo may be directly attachable to the cap. The silo and cap assembly is operably placed over a stoma so as to contain any waste which may unexpectantly exit the stoma. Transparency of the silo allows a user to discern whether/if waste is exiting/exited from a stoma.

In the present embodiment, the cap is of sufficient inside diameter to encase the outer periphery of the stoma at the surface of the user's body (i.e., skin area surrounding the stoma); however, the cap may be configured with a rigid/semi-rigid conical/spherical distal end (i.e., end contacting the skin area) for contacting a portion of the skin area around a stoma. Such a conical/spherical distal end configuration for the cap allows for a larger inner diameter for the cap to enclose a stoma while providing a minimal contact area of the cap with the skin area surrounding the stoma, when the cap is pressed generally axially into the user's flesh.

In an aspect, the cap may include a lip (e.g., flanged area) configured to allow a user to seat the cap while also utilizing the lip of the cap to apply pressure to a portion of the skin area around a stoma. The cap is thereby seated onto the skin area surrounding a stoma to provide sufficient, but minimal, coverage of the skin area surrounding the stoma as the diameter of the cap contact area increases or decreases accordingly with the amount of pressure applied. With the stoma-clean apparatus seated on a minimal skin area surrounding the stoma, a user is able to clean and/or disinfect the skin area around the stoma.

The cap according to the embodiments of the present disclosure, may be constructed of any suitable material and size which allows a user to effectively grasp the stoma-clean apparatus for locating it around a stoma and seating the cap on the skin area surrounding the stoma. In an aspect, the cap may be made of suitable materials including, but not limited to, metals, alloys, or polymers, for example, which may be cleaned and/or disinfected so as to allow for reuse. In another aspect, a cap may be configured, at its distal end (i.e., end contacting the skin area), as a cylindrical, spherical, ellipsoidal, or other suitable shapes such that when the distal end of the cap contacts the skin area around the stoma it forms a seal between the cap and skin area whereby the exposed skin area around the stoma is of minimal size (e.g., diameter). In addition, various shapes and configurations of the stoma-clean apparatus of the present disclosure allows a user to encase the stoma for cleaning the attachment mechanisms by being located on the baseplate and/or flange mechanisms proximal to the stoma. The various shapes and configurations of the cap make the stoma-clean apparatus useful in cleaning stomas located on a user skin area that may be of regular, inward, or outward shape as known in the art.

In yet another aspect, the cap may be constructed of or coated at the distal end with a non-residue, repositionable adhering plastic, silicon, or foam material such as those engineered and manufactured by Adhesives Research or Parafix Engineered Adhesive Solutions. In particular, engineered medical grade open cell foam, such as those engineered by Parafix, used in the construction and/or coating of the distal end of the cap provides an absorptive surface which aids in eliminating waste leakage to the skin area surrounding a stoma.

In yet another aspect, the inside surface of the cap, according to the embodiments of the present disclosure, may be constructed of an absorbent material suitable for capturing body wastes (e.g., defecate, stool, urine, etc.) exiting the stoma and falling toward the user's body.

The stoma-clean apparatus of the foregoing embodiments includes an absorbent, semi-transparent, flexible, lightweight waste collection tube (i.e., silo) for catchment of wastes exiting a stoma. In an example, a user, during the process of changing an ostomy bag with the stoma-clean apparatus in place, will be aware of a stoma's activity by examining such activity—e.g., waste exiting the stoma—through the absorbent, semi-transparent waste collection tube. The absorbent, semi-transparent tube may be comprised of an inner absorbent gauze-like material with a transparent plastic-like outer sheath. The silo may also be constructed (alone or in combination with other materials, such as a sheath) of products such as Vancive™ MED 5580H manufactured by Parafix.

Those of skill in the art will recognize that the stoma-clean apparatus may be configured for use in capturing other waste products or fluids existing the body through various body orifices, including wounds.

In addition to one-piece ostomy appliances (e.g., Hollister Locknroll 1-Piece Drain Pouch), various embodiments of the present disclosure are also be useful for patients wearing two/multi-piece ostomy appliances. In an example, a user of a two-piece ostomy bag, such as the SenSura Mio Convex Flip 2-piece manufactured by Coloplast, includes a bag, baseplate/flange with coupling and lock-ring. The baseplate/flange, coupling, lock-ring, grooves, etc. (i.e., attachment mechanisms) of such devices provide means for attaching the bag, and as such should be cleaned/disinfected as well to avoid bacteria or other contamination build-up in the attachment features, such as grooves, and other mechanisms.

In an aspect, the stoma-clean apparatus of the present disclosure allows a user to encase and hold onto the stoma to allow cleaning of an ostomy appliance's attachment features and mechanisms. A user, having removed a two-piece appliance, attaches a stoma-clean apparatus to the stoma to allow the user to clean the skin area around the stoma. Once the skin area has been cleaned and/or disinfected, the user may attach the baseplate/flange (or the like) to the skin area with the stoma-clean apparatus still secured to the stoma by threading the baseplate/flange over the apparatus' silo and onto the skin area. This action provides further confidence to a user that any inadvertent stoma activity during attachment of the baseplate/flange would be caught in the apparatus. After attachment of the baseplate/flange, the user can easily and swiftly attach the bag portion of the appliance to the baseplate/flange.

According to various embodiments of the present disclosure, an exemplary process for cleaning and disinfecting an area around a stoma for a two/multi-piece ostomy appliance includes:
removing an ostomy bag appliance assembly thereby exposing the skin area around a stoma;
covering and encasing a stoma by attaching a stoma-clean apparatus to the stoma, or alternatively, locating a stoma-clean apparatus by encasing a stoma within the cap portion or annular ring of the apparatus and applying pressure via the cap and/or rigid silo to the area around the stoma;
cleaning/disinfecting the skin area around the stoma; and
attaching an ostomy appliance assembly (e.g., baseplate/flange) to the skin area by threading the appliance assembly over the attached stoma-clean apparatus;
assessing whether a stoma is active via viewing said activity through the transparent/semi-transparent silo, and assuring that the stoma is inactive;
removing the stoma-clean apparatus from the stoma, and attaching an ostomy appliance assembly (e.g., ostomy bag) to the appliance attachment feature(s).

In another aspect, according to various embodiments of the present disclosure, a user may clean and disinfect a one-piece or a two/multi-piece ostomy appliance, where in the case of a two/multi-piece ostomy appliance a baseplate/flange portion remains attached to the user's skin area. An exemplary process for cleaning and disinfecting an area around a stoma includes:
removing an ostomy bag appliance assembly thereby exposing the skin area or appliance feature (e.g., wafer or baseplate/flange) around a stoma;
covering and encasing a stoma by attaching a stoma-clean apparatus to the stoma, or alternatively, locating a stoma-clean apparatus by encasing a stoma within the cap portion or annular ring of the apparatus and applying pressure via the cap and/or rigid silo to the area around the stoma;
cleaning/disinfecting the skin area and/or appliance attachment features around the stoma;
assessing whether a stoma is active via viewing said activity through the transparent/semi-transparent silo and assuring that the stoma is inactive;
removing the stoma-clean apparatus from a non-active stoma and attaching an ostomy appliance assembly or one-piece bag to the ostomy appliance baseplate/flange or skin area, respectively.

While the above exemplary process discloses separate steps for cleaning a one-piece and a two/multi-piece ostomy appliance, it will be understood by those skilled in the art that a user may clean the skin area around a stoma treating a two/multi-piece ostomy appliance as a one-piece assembly.

Other features and aspects of this disclosure will be apparent from the following description and the accompanying drawings.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1A shows a perspective view of a user having a one-piece stoma-clean apparatus located on a stoma, according to an embodiment of the present disclosure;

FIG. 1B shows a perspective view of a stoma exited through an opening of an ostomy appliance baseplate/flange feature; and

FIG. 2 shows an enlarged perspective view of the stoma-clean apparatus of FIG. 1, according to an embodiment of the present disclosure;

FIG. 3A shows a perspective view of a stoma-clean apparatus showing an exemplary cap, according to another embodiment of the present disclosure;

FIGS. 3B-3D show perspective views of exemplary caps of a stoma-clean apparatus, according to various embodiments of the present disclosure;

FIG. 4A is a front view of a stoma-clean apparatus showing an exemplary conical/spherical cap, according to another embodiment of the present disclosure;

FIG. 4B is a front view of a stoma-clean apparatus showing an exemplary conical/spherical cap with a lip, according to another embodiment of the present disclosure;

FIGS. 5 and 6 details exemplary processes for using a stoma-clean apparatus, according to various embodiments of the present disclosure; and

FIGS. 7A and 7B show a side elevational view and a sectional view, respectively of an exemplary stoma-clean apparatus, according to another embodiment of the present disclosure.

### DETAILED DESCRIPTION

Reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

The detailed description describes non-limiting exemplary embodiments. Any individual features may be combined with other features as required by different applications for at least the benefits described herein. As used herein, for example, the terms “proximal” refers to parts closer to a relevant feature, while “distal” refers to parts away from a relevant feature unless specifically indicated otherwise.

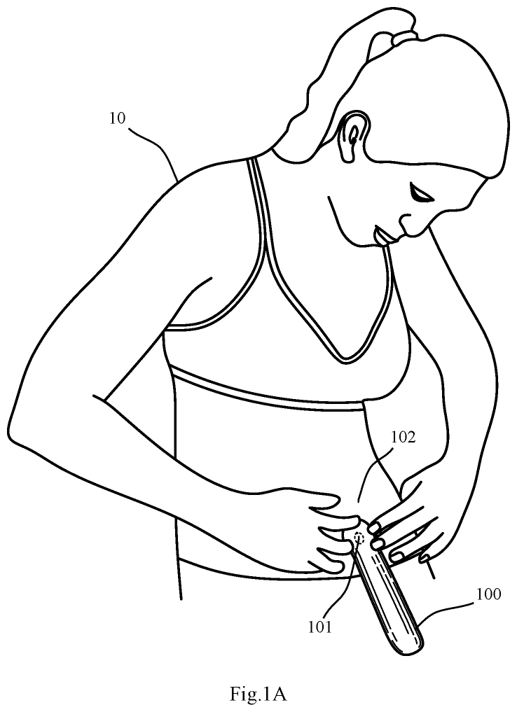

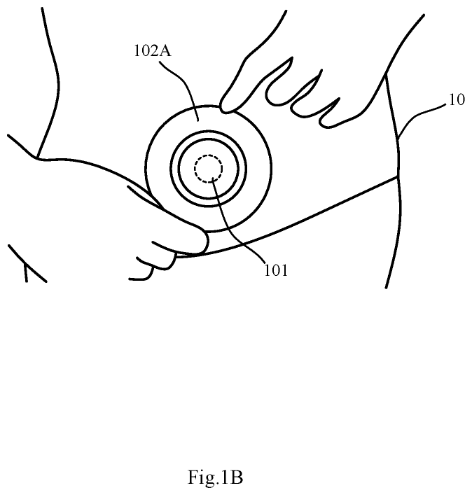

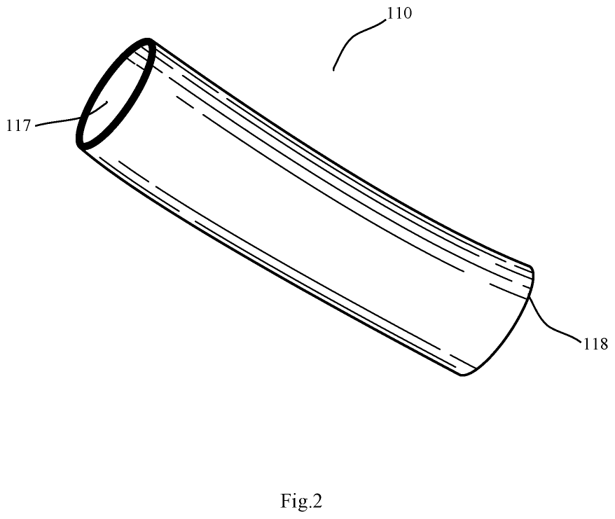

FIGS. 1A and 1B illustrate perspective views of a user 10 with a stoma-clean apparatus 100 releasably held onto an exposed stoma 101 in proximity to the user's skin area 102, according to an embodiment of the present disclosure. By reference to FIG. 2, the exemplary one-piece stoma-clean apparatus 110 includes an absorbent, semi-transparent, flexible, lightweight waste collection tube (i.e., silo) 112. The silo 112 includes an open end 117, a closed end 118, wherein the open end 117 is elasticized to allow hands-free attachment of the stoma-clean apparatus 110 to the stoma. The one-piece stoma-clean apparatus 110 may be constructed from moldable, absorbent, and transparent medical grade materials such as those engineered by 3M, Adhesives Research, and Parafix Engineered Adhesives Solutions.

In an alternative embodiment, the open end 117 of the stoma-clean apparatus 100 may be substantially configured with an elastic annular seal ring 115 (not shown). The seal ring 115 is disposed on, or integrated with, the open end 117 of the silo 112. The annular seal ring 115 may be elasticized or constructed of a moldable wafer to assist the user 10 in holding the stoma-clean apparatus 100 onto the stoma 101. In an exemplary aspect, the annular seal ring 115 may be configured as an adjustable diameter clasp or other mechanism allowing adjustability of the inside diameter of the annular seal ring 115 to allow a user to attach the stoma-clean apparatus 100 to the stoma 101 in a hands-free manner.

To contain any waste exiting the stoma 101, a user 10 attaches the stoma-clean apparatus 110 substantially over the stoma 101 so as to cover and encase the active portion of the stoma 101 as shown in FIG. 1A. The stoma-clean apparatus 110 is held in-place on the stoma 101 by the elasticized open end 117 whereby the open end 117 is held proximal to the skin area 102. With the stoma-clean apparatus 110 situated hands-free on the stoma 101, a user 10 is able to clean the skin area 102 around the periphery of the open end 117 of the stoma-clean apparatus 110.

In an alternative embodiment, to contain waste exiting the stoma 101, a user 10 places the seal ring 115 over the stoma 101 and seats the ring 115 onto the skin area 102 around the stoma 101 thereby containing the stoma 101 in the stoma-clean apparatus 110. The stoma-clean apparatus is held in-place on the skin area 102 with one hand while the user 10 cleans the skin area 102 around the periphery of the seal ring 115 with the other hand. In another aspect, the annular seal ring 115 may be elasticized or constructed of a moldable wafer to assist the user 10 in holding the stoma-clean apparatus 100 onto the stoma 101. In an exemplary embodiment, the annular seal ring 115 may be configured as an adjustable diameter clasp or other mechanism allowing adjustability of the inside diameter of the annular seal ring 115.

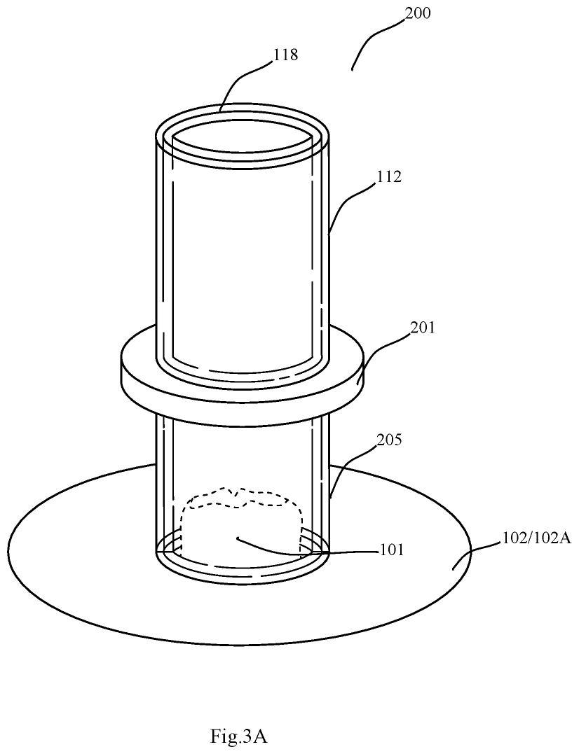

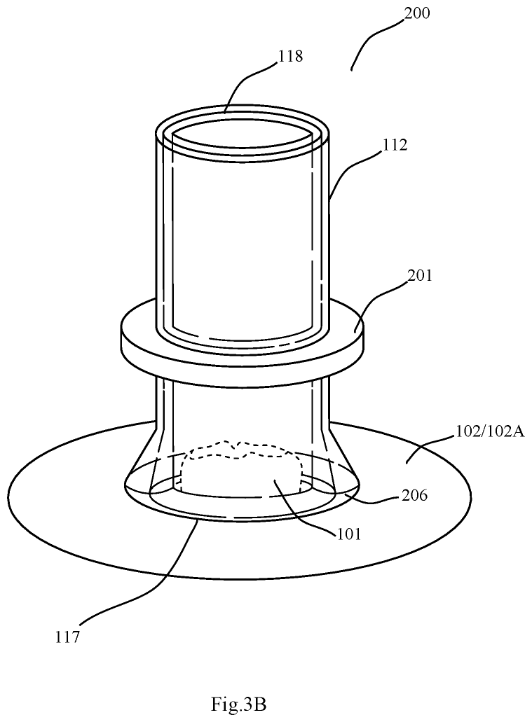

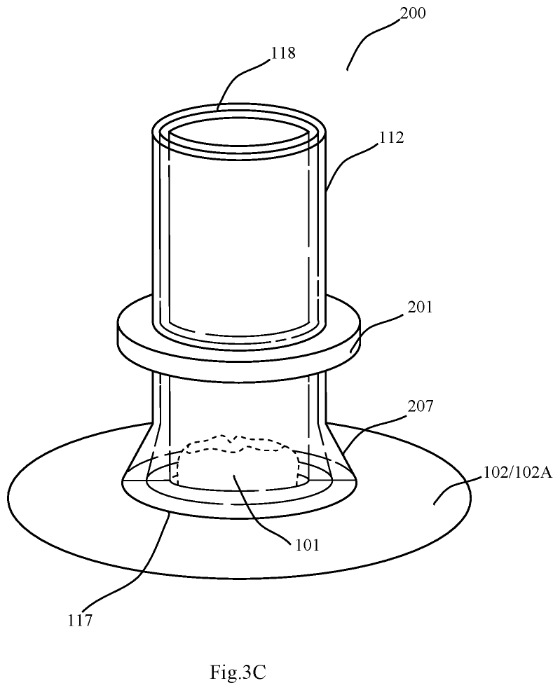

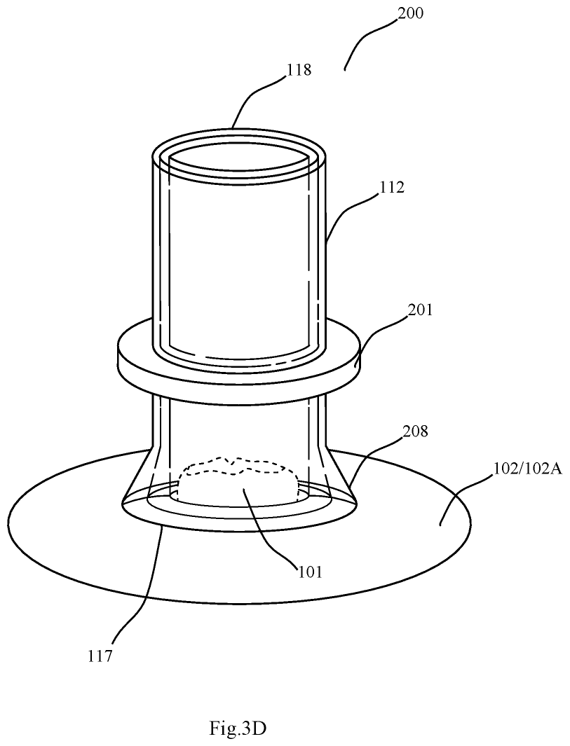

By reference to FIG. 3A, according to an exemplary embodiment of the present disclosure, a stoma-clean apparatus 200 includes an absorbent, semi-transparent, rigid/semi-rigid silo 112 with a closed end 118, an open end 117, and a cap 205. The cap 205 is shaped as a cylindrical rigid body with a thru-hole configured to enclose a stoma 101. In an aspect, the cap 205 may be of another shape such as an elbow while still enabling enclosure of the stoma 101. The stoma-clean apparatus 200 also includes a coupling ring 201 for attaching the silo 112 to the cap 205. In an aspect, a stoma-clean apparatus may be constructed without utilizing a coupling ring 201 such as by directly fusing/molding the silo 112 to the cap 205.

FIGS. 3B-3D illustrate other caps 206, 207, and 208 according to various exemplary embodiments of the present disclosure. In an aspect, cap 206 is configured with a convex flange at its distal end which is useful for contacting a user's surrounding skin area which is inward in shape. Likewise, cap 207 is configured with a straight flange at its distal end which is useful for contacting a user's surrounding skin area which is regular (i.e., flat) in shape. Similarly, cap 208 is configured with a concave flange at its distal end which is useful for contacting a user's surrounding skin area which is outward in shape. However, it is to be understood that the shape of the caps in the stoma-clean apparatus 200 is not limiting the scope of the present disclosure.

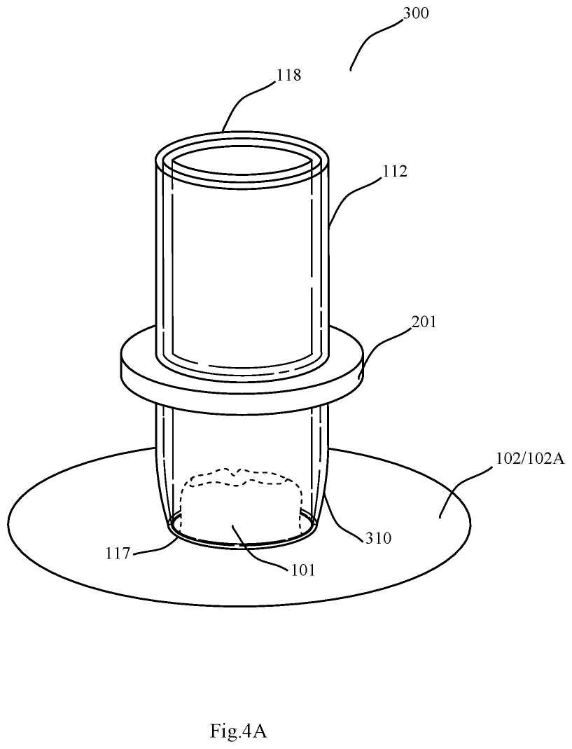

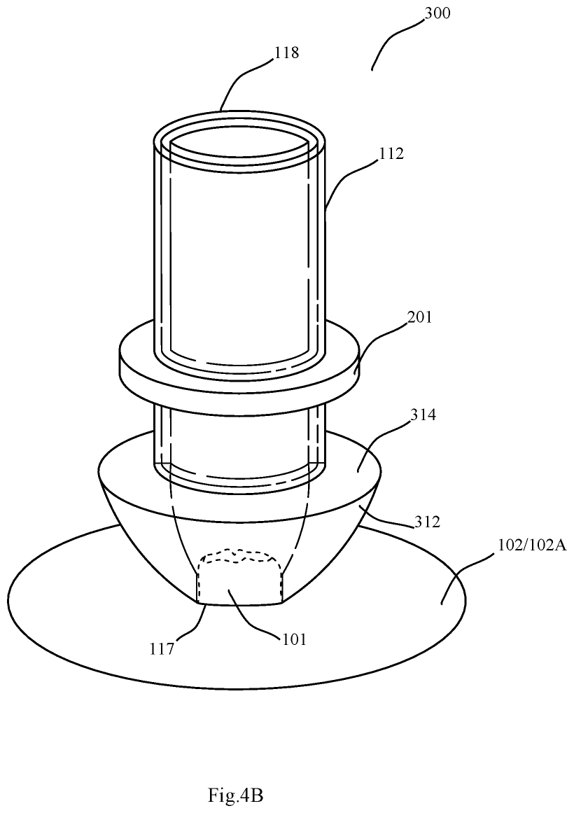

In yet another embodiment of the present disclosure, a stoma-clean apparatus 300 includes an absorbent, semi-transparent, rigid/semi-rigid silo 112 with a closed end 118 and an open end 117, a conical/spherical cap 310, and a coupling ring 201 for attaching the silo 112 to the conical/spherical cap 310 as shown in FIG. 4A. The cap 310 is configured with a rigid/semi-rigid conical/spherical end for contacting a portion of the skin area 102 around a stoma 101 and provides a smaller diameter “D_{C}” around the stoma peripheral diameter “D_{S}” proximal to the user's skin area 102. The conical/spherical configuration of exemplary cap 310 enables a user 10 to clean the skin area 102 closer to the stoma peripheral diameter “D_{S}” and thereby affords a user the ability to clean a larger surface skin area 102 surrounding the stoma 101. In an exemplary aspect, conical/spherical cap 312 (as shown in FIG. 4B) includes a lip 314 configured thereon to allow a user to seat the stoma-clean apparatus on the user's skin area 102 with greater stability while also utilizing cap 312 configured with the lip 314 to apply pressure to a portion of the skin area 102 around a stoma.

By reference to FIG. 4B, the exemplary cap 312 includes a lip 314 configured on its surface generally perpendicular to the longitudinal axis “A-A” of cap 312. Cap 312 may have lip 314 installed anywhere on its surface and in any angular orientation “∅0” with respect to longitudinal axis “A-A”. Lip 314 may be integrally constructed on cap 312, such as by molding, or separately affixed to cap 312 using known construction methods (e.g., adhesively bonded).

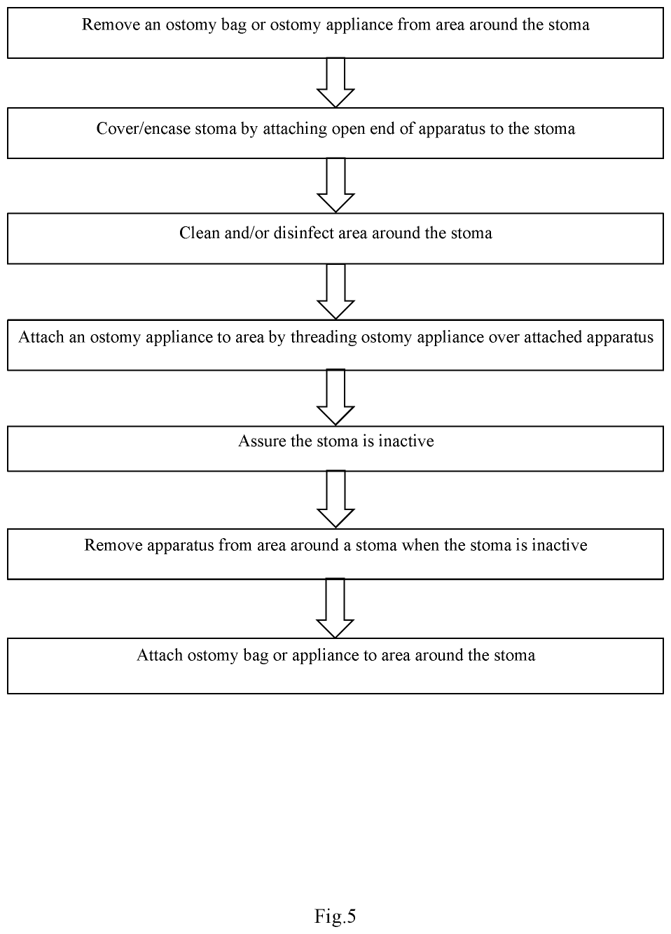

In the case of a two/multi-piece ostomy appliance, FIG. 5 illustrates a flow chart of a method 600 using a stoma-clean apparatus 110, 200, 300, 400 for cleaning and disinfecting an area around a stoma. The sequence in which the method 600 is described is not intended as a limitation, and any number of the described method steps may be combined in any order to implement the method, or an alternative method. Additionally, individual blocks may be deleted from the method without departing from the spirit and scope of the present disclosure.

At step 602, the method 600 includes removing an entire ostomy bag appliance from the body of a user 10 thereby exposing the user's skin area 102 around a stoma 101.

At step 604, the method 600 includes covering/encasing a stoma 101 by attaching a stoma-clean apparatus 110, 200, 300, 400 to the stoma 101. Alternatively, in accordance with other embodiments of the present disclosure, the method 600 includes a user 10 encasing a stoma 101 within the stoma-clean apparatus 110, 200, 300, 400 (for example, within annular seal ring 115, cap 205 and/or silo 112, etc.) and applying pressure via the ring, cap, and/or silo (for example, via the annular seal ring 115, cap 205 and/or silo 112, etc.) to the skin area 102 around the stoma 101.

At step 606, the method 600 includes a user 10 cleaning and/or disinfecting the skin area 102 around the stoma 101.

At step 608, the method 600 includes a user 10 attaching an ostomy bag appliance assembly (e.g., baseplate/flange) to the skin area 102 by threading the appliance assembly over the attached stoma-clean apparatus 110.

At step 610, the method 600 includes a user 10 assessing whether a stoma 101 is active via viewing said activity through the transparent/semi-transparent silo 112.

At step 612, the method 600 includes removing the stoma-clean apparatus 110, 200, 300, 400 from a non-active stoma.

At step 614, the method 600 includes, attaching an ostomy bag onto the user's skin area 102 or ostomy bag appliance attachment feature 102A, around the stoma 101.

In an exemplary aspect, an ostomy bag may be of a two-piece configuration such as those known in the art and discussed above. Where a baseplate, or the like, remains attached to a user's skin area 102 after removal of an ostomy bag, a stoma-clean apparatus 110, 200, 300, 400 would be used to cooperate with the surface of the device baseplate/flange versus directly onto a user's skin area 102 without departing from or limiting the essential scope or teachings of the present disclosure.

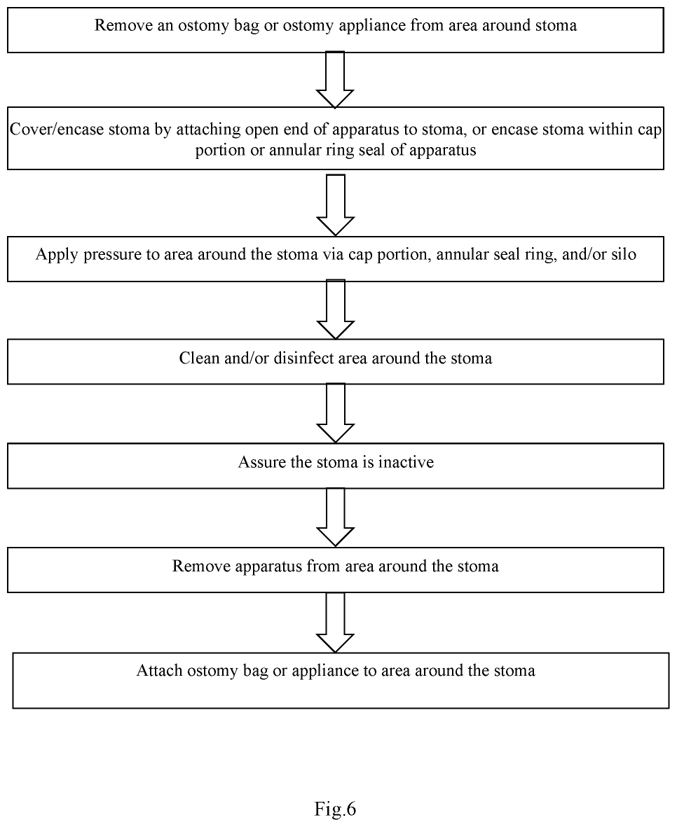

For a one-piece or two/multi-piece ostomy appliance, where in the case of a two/multi-piece ostomy appliance a wafer or baseplate/flange portion remains attached to the user's skin area, FIG. 6 illustrates a flow chart of a method 700 for using a stoma-clean apparatus 110, 200, 300, 400 for cleaning and disinfecting an area 102/102A around a stoma 101. The sequence in which the method 700 is described is not intended as a limitation, and any number of the described method steps may be combined in any order to implement the method, or an alternative method. Additionally, individual blocks may be deleted from the method without departing from the spirit and scope of the present disclosure.

At step 702, the method 700 includes removing an ostomy bag, such as a one-piece bag, or an ostomy appliance, such as a two-piece bag with a wafer and/or flanged assembly (See FIG. 1B) thereby exposing an area around a stoma 101 for cleaning.

At step 704, the method 700 includes covering and encasing a stoma 101 by attaching a stoma-clean apparatus 110, 200, 300, 400 to the stoma 101. locating a stoma-clean apparatus 110, 200, 300, 400 by encasing a stoma 101. Alternatively, in accordance with other embodiments of the present disclosure, the method 700 includes a user 10 encasing a stoma 101 within the stoma-clean apparatus 110, 200, 300, 400 (for example, within annular seal ring 115, cap 205 and/or silo 112, etc.) and applying pressure via the ring, cap, and/or silo (for example, via the annular seal ring 115, cap 205 and/or silo 112, etc.) to the area 102/102A around the stoma 101.

At step 706, the method 700 includes a user 10 cleaning/disinfecting the area 102/102A around the stoma 101.

At step 708, the method 700 includes a user 10 assessing whether a stoma 101 is active via viewing said activity through the transparent/semi-transparent silo 112.

At step 710, the method 700 includes removing the stoma-clean apparatus 110, 200, 300, 400 from a non-active stoma and attaching an ostomy appliance assembly or one-piece bag to the area 102/102A.

In an exemplary aspect, an ostomy device may be a two-piece configuration such as those known in the art and discussed above. Where a baseplate, or the like, remains attached to a user's skin area 102 after removal of an ostomy bag, a stoma-clean apparatus 110, 200, 300, 400 would be used to cooperate with the surface of the device baseplate/flange area 102A versus directly onto a user's skin area 102 without departing from or limiting the essential scope or teachings of the present disclosure.

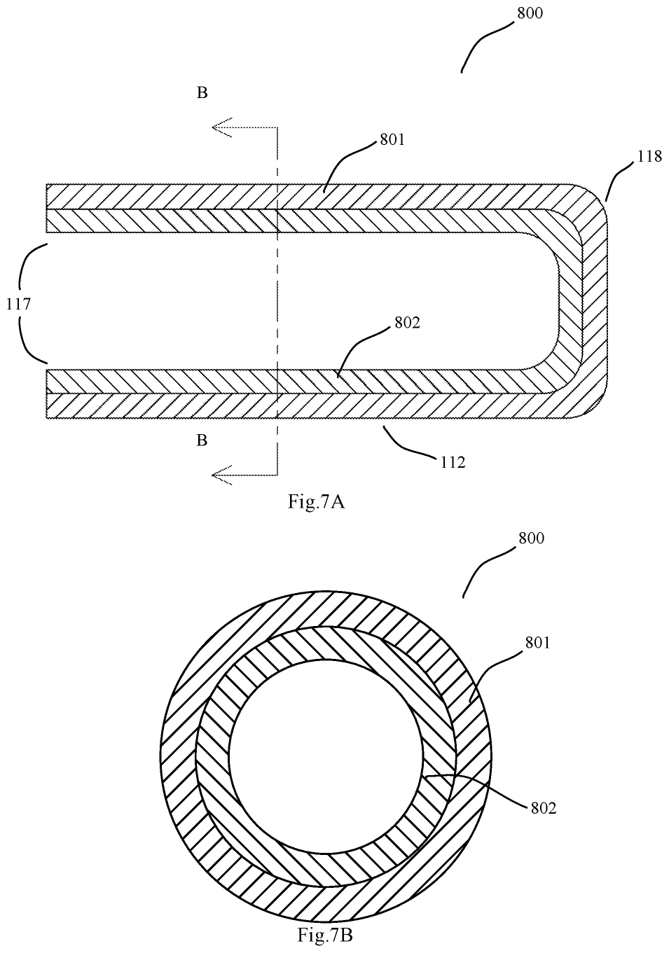

FIGS. 7A and 7B illustrate views of an exemplary stoma-clean apparatus 800 according to yet a further embodiment of the present disclosure. A stoma-clean apparatus 800, including an open end 117, a closed end 118, and a silo 112, may be constructed by layering and/or adhering a moisture resistant transparent/semi-transparent sheath 801 onto an absorbent, semi-transparent layer 802 comprised of an absorbent material such as gauze. The stoma-clean apparatus 800 and, independently, layers 801 and 802 may be flexible, semi-rigid, or rigid. Those of skill in the art will recognize that the stoma-clean apparatus 800 may be constructed using engineered materials (e.g., Vancive™ MED 5580H) such as those engineered and manufactured by Adhesives Research or Parafix Engineered Adhesive Solutions, for example.

The exemplary stoma-clean apparatus 800 includes features and performance aspects similar to that of stoma-clean apparatus 110, without limitation, such as an elasticized open end 117.

While the disclosure has been described with reference to one or more different exemplary embodiments, it will be understood by those skilled in the art that various changes may be made and equivalents may be substituted for elements thereof without departing from the scope of the disclosure. For example, in some cases reference to application of a stoma-clean apparatus to a user's skin area may similarly include application of the stoma-clean apparatus to the exposed area of an ostomy device (e.g., wafer, flange, etc.). In addition, many modifications may be made to adapt to a particular situation without departing from the essential scope or teachings thereof. Therefore, it is intended that the disclosure not be limited to the particular embodiment disclosed as the best mode contemplated for carrying out this invention.

### LIST OF ELEMENTS

10 User

100 Stoma-clean apparatus

101 Stoma

102 Skin area

110 Stoma-clean apparatus

112 Waste collection tube, Silo

115 Seal ring

117 Open end

117A Open end, seal ring

118 Closed end

200 Stoma-clean apparatus

201 Coupling ring

205 Cylindrical cap

206 Convex-flanged cap

207 Straight-flanged cap

208 Concave-flanged cap

300 Stoma-clean apparatus

310 Conical/Spherical cap

312 Conical/Spherical cap with lip

314 Lip

600 Method

602 Step

604 Step

606 Step

608 Step

610 Step

700 Method

702 Step

704 Step

706 Step

708 Step

710 Step

800 Stoma-clean apparatus

801 Sheath

802 Absorbent layer

A-A Longitudinal axis of cap

D_{C} Diameter at contact area

D_{S} Stoma periphery diameter

∅ Angle

What is claimed is:

1. An apparatus for restraining and/or containing a stoma, said apparatus adapted to aid cleaning and disinfecting an area around the stoma, the apparatus comprising:
a silo, said silo including a hole, and said silo having an open end and a closed end, and
a seal ring or a cap, said seal ring or cap having a predetermined shape adapted to couple with the open end of the silo and encase the stoma.

2. The apparatus of claim 1, further comprising:
the seal ring or cap having an adjustable and/or moldable inner diameter adapted for releasably attaching the seal ring or cap to the stoma, and wherein
the silo is comprised of an absorbent transparent or semi-transparent material configured to capture waste exiting the stoma.

3. The transparent or semi-transparent material of claim 2 comprising a gauze inner layer and a leak-proof transparent outer layer.

4. The apparatus of claim 1, further comprising:
the cap comprised of a cylindrical shaped semi-rigid material with a hole for allowing waste to pass therethrough the hole to the silo, and wherein
the inner diameter of the cap is comprised of a moldable and/or adjustable inner diameter adapted for releasably attaching the inner diameter of the cap to the stoma, and wherein
the silo is comprised of an absorbent transparent or semi-transparent material configured to capture waste exiting the stoma.

5. The silo of claim 2 wherein the absorbent transparent or semi-transparent material is of a semi-rigid or rigid construction.

6. The cap of claim 1, further comprising a flanged end adapted for contacting the skin area of a user, and wherein the flanged end is straight, concave, or convex in shape.

7. An apparatus for restraining and/or containing a stoma, said apparatus adapted to aid cleaning and disinfecting an area around a stoma, the apparatus comprising:
a transparent or semi-transparent silo or silo portion, said silo or silo portion including a hole, and said silo or silo portion having an open end and a closed end, wherein said open end is elastic.

8. The transparent or semi-transparent material of claim 7 comprising a gauze inner layer and a leak-proof transparent outer layer.

9. The silo of claim 7 wherein the absorbent transparent or semi-transparent material is of semi-rigid or rigid construction.

10. The apparatus of claim 7, being of one-piece construction, said apparatus further comprising an absorbent transparent or semi-transparent material.

11. A method, including an apparatus for restraining and/or containing a stoma adapted for cleaning and disinfecting an area around the stoma, the apparatus comprising a transparent or semi-transparent silo or silo portion, said silo or silo portion including a hole, and said silo or silo portion having an open end and a closed end, wherein said open end is elastic, the method comprising:
removing an ostomy bag or appliance, thereby exposing the area around the stoma;
covering and encasing the stoma by attaching the open end of the apparatus to the stoma;
cleaning and/or disinfecting the area around the stoma;
attaching an ostomy appliance wafer, baseplate, or flange assembly by threading said ostomy appliance wafer, baseplate, or flange over the attached apparatus;
viewing through the transparent or semi-transparent silo or silo portion, thereby assuring the stoma is inactive;
removing the apparatus from the area, wherein the stoma is determined to be inactive; and
attaching an ostomy bag to the area around the stoma.

12. A method, including an apparatus for restraining and/or containing a stoma, said apparatus adapted to aid cleaning and disinfecting an area around the stoma, the apparatus comprising:
a silo, said silo including a hole, and said silo having an open end and a closed end, and
a seal ring or a cap, said seal ring or cap having a predetermined shape adapted to couple with the open end of the silo and encase the stoma, the method comprising:
removing an ostomy bag or appliance, thereby exposing the area around the stoma;
covering and encasing the stoma by attaching the open end of the apparatus to the stoma, or encasing a stoma within the cap portion or annular ring of the apparatus and applying pressure to the area around the stoma via the cap and/or silo;
cleaning and/or disinfecting the area around the stoma;
viewing through the transparent or semi-transparent silo or silo portion, thereby assuring the stoma is inactive;
removing the apparatus from the area, wherein the stoma is determined to be inactive; and
attaching an ostomy bag or appliance to the area around the stoma.
